(19) United States
(12) Patent Application Publication
(10) Pub. No.: US 2006/0123664 A1
(43) Pub. Date: Jun. 15, 2006

(54) Insole having multiple energy sources

(76) Inventors: Robert E. Boyd, Hudson, OH (US); James A. Forsyth, Stow, OH (US)

Correspondence Address: Theodore D. Lienesch;Thompson Hine LLP, 2000 Courthouse Plaza NE, 10 West Second Street, Dayton, OH, 45402-1758, US

(21) Appl. No.: 10/686,353

(22) Filed: Oct. 14, 2003

(51) Int. Cl. A43B 13/38; A61F 5/14

(52) U.S. Cl. 36/44; 36/180

(57) ABSTRACT

A shoe insole (or sole) includes at least two elastomeric pads horizontally distinct from one another, each pad having a different rebound rate. The insole thereby provides at least one pad with relatively greater shock absorbing properties and at least one other pad with relatively greater energy return, or lift, properties. These differing characteristics allow an insole to be customized with regard to the specific use of a shoe.

### BACKGROUND OF THE INVENTION

1. Technical Field

The invention relates to an improved insole for a shoe. More particularly, the invention relates to an insole having impact-absorbing properties as well as energy return properties. Specifically, the invention relates to an insole having at least two elastomers each having different rates of energy absorbing and/or rebound properties.

2. Background Information

Many thousands of insoles have been designed for shoes to increase the comfort as well as the performance for various activities, especially different sports such as running, baseball, football, tennis and so forth. Each type of activity requires the foot to perform in a different way and thus insoles have been designed to aide in particular sports as well as in a general manner whereby shock absorbing properties and the like are improved. Various insoles have used liquid-containing cavities to provide improved shock absorption and comfort to the foot, for example, U.S. Pat. No. 3,724,106, U.S. Pat. No. 4,115,934, U.S. Pat. No. 4,471,538, U.S. Pat. No. 5,155,927, and U.S. Pat. No. 5,979,086. Other insoles have used air-filled chambers, such as U.S. Pat. No. 5,996,253. Some insoles have employed various structural features to provide cushioning, such as U.S. Pat. No. 5,642,575, which utilizes domes of resilient material for that purpose. Still other insoles use cushioning elements which are placed in various areas of the insole, such as the heel and the ball area of the foot. For instance, U.S. Pat. No. 6,176,025 utilizes such cushioning elements, whereby a cushioning element is made of a polymeric foam which is firmer than the surrounding polymeric foam of the remainder of the insole. Alternately, the patent uses a cushioning element using a gel-air mixture whereby an air cushioning pad is surrounded by an outer cushioning ring and the cushioning pad has a lower density than the outer ring.

However, there remains a need for an insole having separate portions made of materials having different rebound rates, such that one section primarily absorbs impact energy while another portion returns energy to the foot after impact. While this concept is useful in a variety of configurations, it is perhaps most easily understood in regard to the motion of a runner, whereby it is desired to have a greater shock absorbing property in the heel as the runner's shoe impacts with the ground, along with a high energy return rate, or rebound rate, in the ball area of the foot to provide extra spring to the front of the foot as the runner propels himself forward.

### BRIEF SUMMARY OF THE INVENTION

The present invention provides an insole having a first elastomeric pad and a second elastomeric pad horizontally distinct from one another, each pad having a rebound rate; and the rebound rate of the first pad differing from the rebound rate of the second pad.

The present invention provides a shoe having a sole comprising a first elastomeric pad and a second elastomeric pad horizontally distinct from one another, each pad having a rebound rate; and the rebound rate of the first pad differing from the rebound rate of the second pad.

### BRIEF DESCRIPTION OF THE SEVERAL VIEWS OF THE DRAWINGS

FIG. 1 is a bottom plan view of a first embodiment of the present invention.

FIG. 2 is an exploded bottom plan view of the first embodiment.

FIG. 3 is a top plan view of the first embodiment.

FIG. 4 is a sectional view taken on line 4-4 of FIG. 1.

FIG. 5 is a sectional view taken on line 5-5 of FIG. 1.

FIG. 6 is a bottom plan view of a second embodiment of the present invention.

FIG. 7 is a sectional view taken on line 7-7 of FIG. 6.

FIG. 8 is a sectional view taken on line 8-8 of FIG. 6.

FIG. 9 is a sectional view of a third embodiment of the present invention.

FIG. 10 is a sectional view of a fourth embodiment of the present invention.

### DETAILED DESCRIPTION OF THE INVENTION

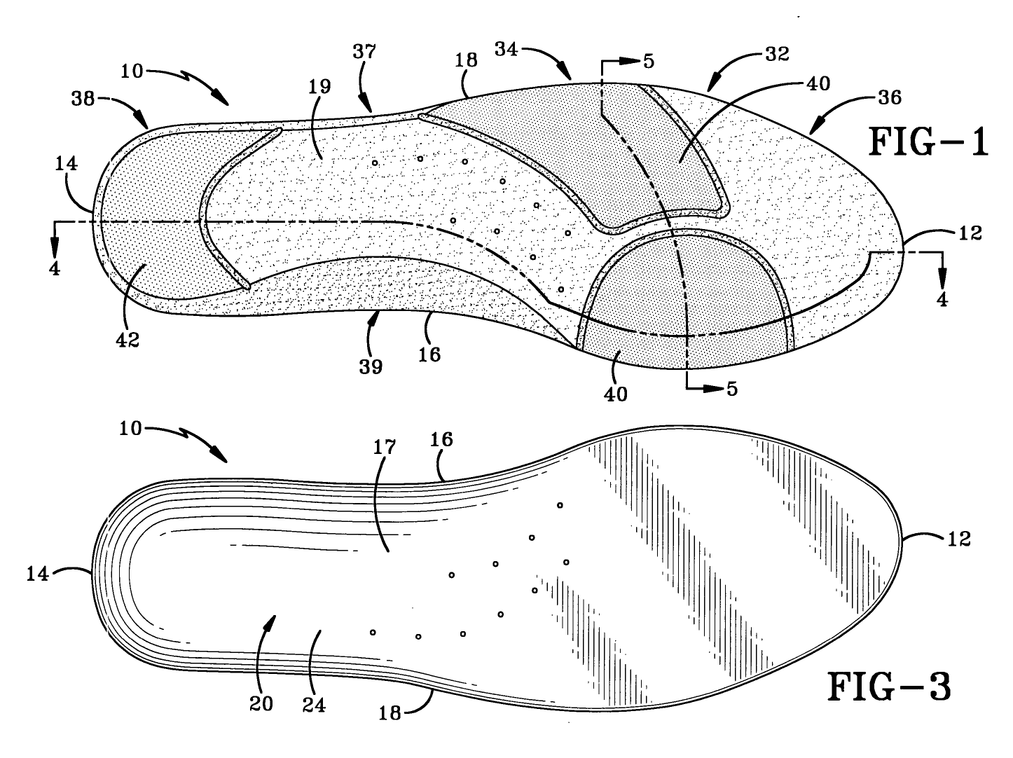

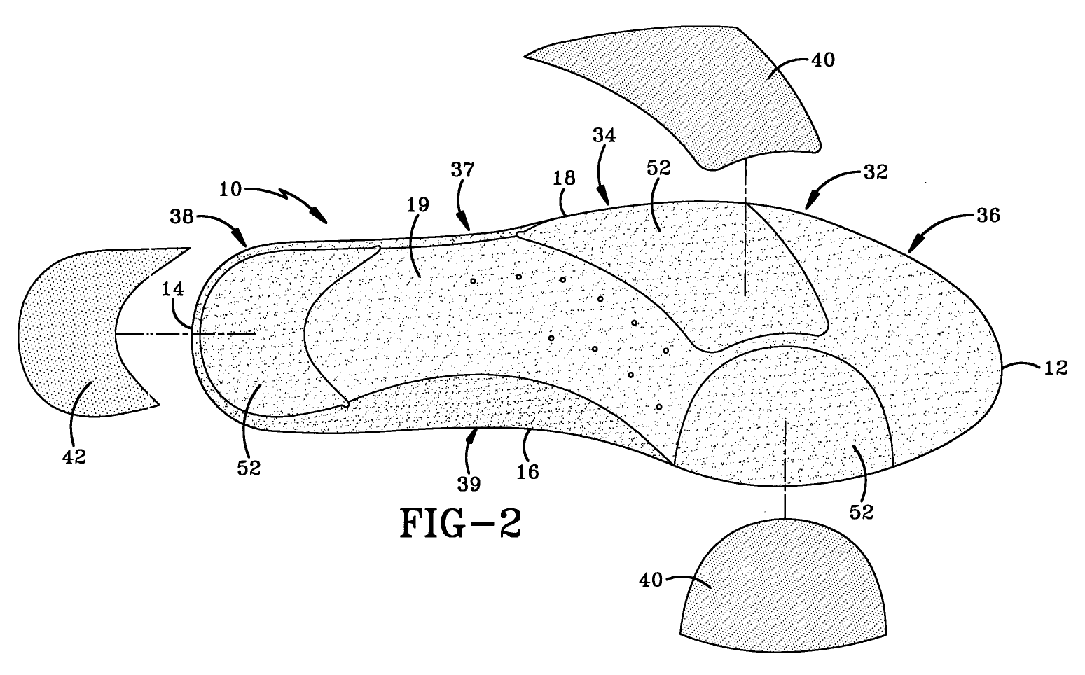

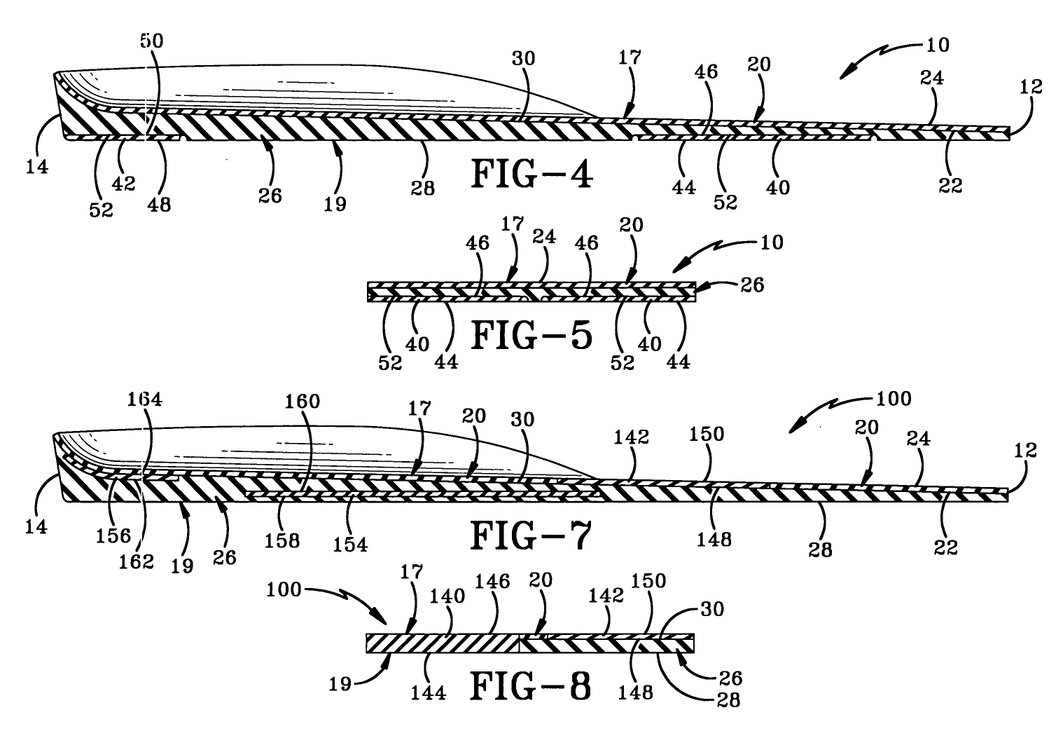

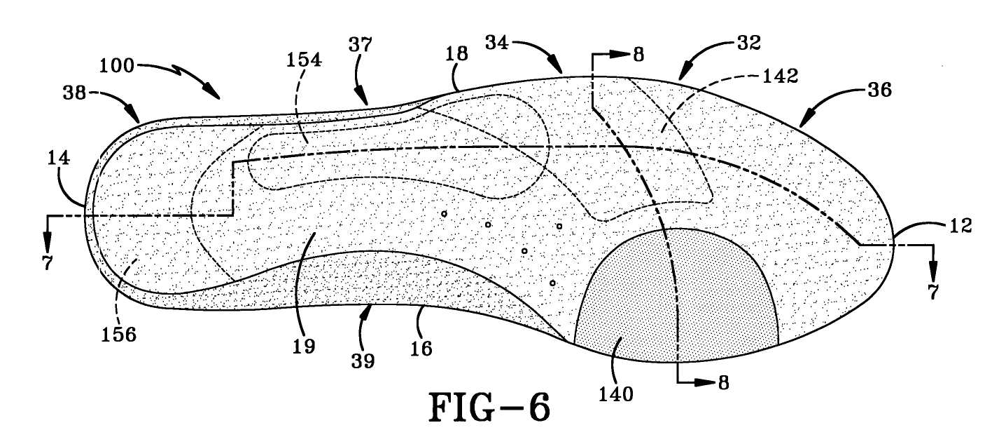

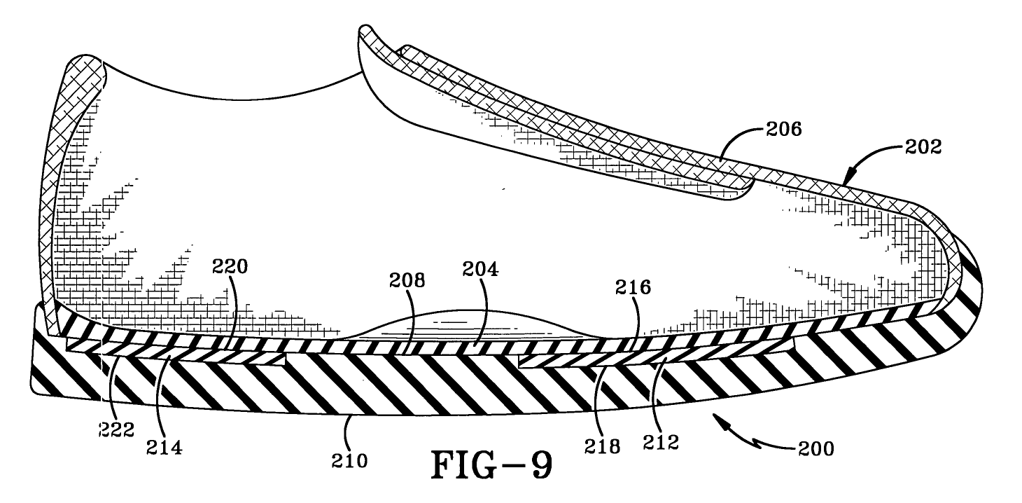

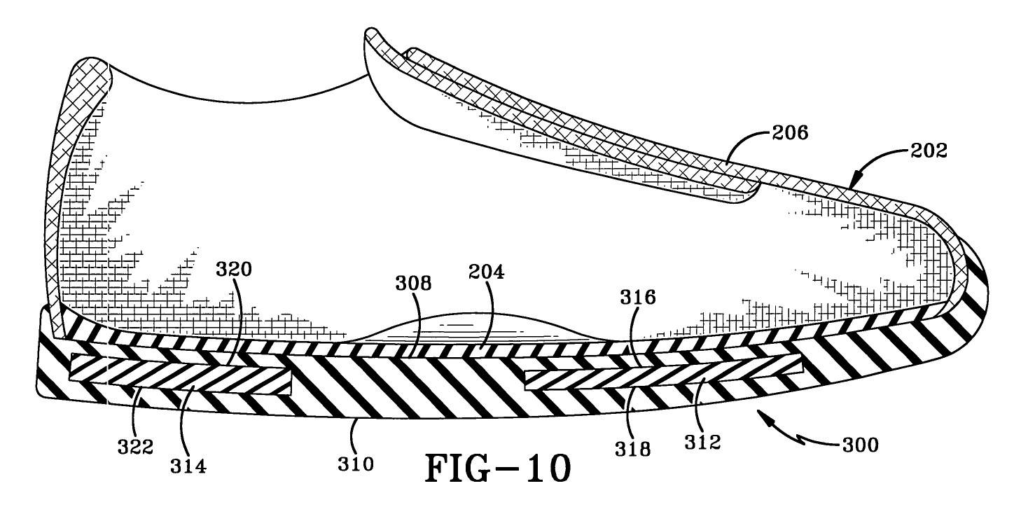

A first embodiment of the insole of the present invention is indicated generally at 10 and is shown particularly in FIGS. 1-5. A second embodiment of insole of the present invention is indicated generally at 100 in FIGS. 6-8. A third embodiment of the invention applies the concept to a sole of a shoe and is shown generally at 200 in FIG. 9. A fourth embodiment similar to the third embodiment is indicated generally at 300 in FIG. 10.

Insole 10 has a toe or forward end 12, a heel or rearward end 14, and inner edge 16 situated along the arch side of insole 10 and extending generally the length thereof and an outer edge 18 opposite inner edge 16 which extends generally along the length of insole 10. Insole 10 has an upper surface 17 and a lower surface 19. Referring to FIGS. 4-8, insole 10 also includes an upper layer 20 having a lower surface 22 and an upper surface 24 upon which a foot rests when insole 10 is in use. Upper surface 17 of insole 10 coincides with upper surface 24 of upper layer 20. Insole 10 further includes a lower layer 26 having a lower surface 28 and an upper surface 30 to which upper layer 20 is attached along lower surface 22 thereof. Upper layer 20 and lower layer 26 are each preferably formed of a polymeric foam material with upper surface 24 of upper layer 20 preferably being of a particularly durable quality to endure the various stresses received from the foot of the user. However, upper layer 20 and lower layer 26 can be made of a variety of materials as known in the art without departing from the spirit of the invention. Referring specifically to FIG. 6, insole 10 may be further broken down into a forefoot section 32, which generally comprises a metatarsus section 34 and a toe section 36, along with a midfoot section 37 situated rearwardly of forefoot section 32 and a heel section 38 rearward of midfoot section 37. Midfoot section 37 includes an arch section 39 along inner edge 16. Insole 10 further includes a first pad 40 divided into two side-by-side separated pieces in the general area of metatarsus section 34 and a second pad 42 in the general area of heel section 38. First pad 40 may also be a single continuous piece or more than two pieces without departing from the spirit of the invention. First pad 40 has a lower surface 44 and an upper surface 46 to which lower layer 26 is attached along lower surface 28 thereof. Second pad 42 likewise has a lower surface 48 and an upper surface 50 to which lower layer 26 is attached along lower surface 28 thereof. Lower surface 28 of lower layer 26, lower surface 44 of first pad 40 and lower surface 48 of second pad 42 are all generally on the same level. This is due to the fact that in the areas of first pads 40 and second pad 42, lower layer 26 is recessed approximately the thickness of the respective pads 40 and 42. The depth of these recessed areas 52 may vary and may even be eliminated without departing from the spirit of the invention. Alternate locations for first pad 40 and second pad 42 are hereafter more fully indicated in regard to the second embodiment.

In accordance with one of the main features of the present invention, first pad 40 is horizontally distinct from second pad 42 and each pad is made of an elastomer having a different rebound rate. The term horizontally distinct in the exemplary embodiment means that the two pads 40 and 42 are physically separated with horizontal space therebetween. However, without departing from the spirit of the invention, horizontally distinct may also mean, for example, that the two pads 40 and 42 lie side-by-side in contact with one another or that the two pads 40 and 42 partially overlap one above the other as long as there are still two horizontally distinct portions having different rebound rates. In the first embodiment, the rebound rate of first pad 40 is greater than the rebound rate of second pad 42. Thus, as for use with a runner, second pad 42 absorbs a greater amount of energy from the impact of a foot coming downwardly thereon in comparison to first pad 40, which returns a relatively greater amount of the impact energy to the foot to help it spring upward and forward. In more common terms, second pad 42 tends to absorb or deaden the impact from the generally downward movement of the foot while first pad 40 relatively bounces back or returns the generally downward impact energy to create an upward lift.

In accordance with another main feature of the present invention, the characteristics of the elastomers of first pad 40 and second pad 42 are as follows. The elastomer used in first pad 40 has a rebound rate ranging from 5.0% to 90.0%, a hardness with a durometer range of 30 to 70, Shore Hardness (00) scale, and a density ranging from 0.8 to 2.2. The elastomer of second pad 42 has a rebound rate ranging from 1.0% to 40.0%, a hardness with a durometer range or 20 to 70, Shore Hardness (00) scale, and a density ranging from 0.7 to 2.0. Preferably, the polymer of first pad 40 has a hardness with a durometer range of 35 to 70, Shore Hardness (00) scale while the polymer of second pad 42 preferably has a hardness with a durometer range of 20 to 55, Shore Hardness (00) scale.

FIGS. 6-8 show the second embodiment, insole 100. Insole 100 shows some of the endless configuration possibilities of the present invention and is numbered like insole 10 except for the pads and their relative locations as noted herein. As shown in FIG. 6, insole 100 includes a first pad 140 positioned generally in metatarsus section 34 along inner edge 16; a second pad 142 positioned in metatarsus section 34 along outer edge 18 adjacent but slightly spaced from pad 140; a third pad 154 positioned in midfoot section 37 along outer edge 18 and partially overlapping with second pad 142; and a fourth pad 156 positioned in heel section 38.

FIGS. 7 and 8 show the vertical position, in addition to the horizontal position, of each pad 140, 142, 154 and 156. First pad 140 has a lower surface 144 and an upper surface 146. Second pad 142 has a lower surface 148 and an upper surface 150. Third pad 154 has a lower surface 158 and an upper surface 160. Fourth pad 156 has a lower surface 162 and an upper surface 164. First pad 140 (FIG. 8) extends from upper surface 17 to lower surface 19 of insole 100 so that upper surface 146 and lower surface 144 of first pad 140 coincide respectively with upper surface 17 and lower surface 19 in the area of first pad 140. Second pad 142 is generally in a side-by-side or lateral orientation with respect to first pad 140 and is inserted into upper layer 20 of insole 100. Upper surface 150 and lower surface 148 coincide respectively with upper surface 24 and lower surface 22 of upper layer 20 in the area of second pad 142. Upper surface 150 also coincides with upper surface 17 of insole 100 in the area of second pad 142. Third pad 154 is situated entirely within lower layer 26. Thus, lower surface 158 and upper surface 160 of third pad 154 are positioned between lower surface 28 and upper surface 30 of lower layer 26. Further, the portion of third pad 154 which overlaps with the respective portion of second pad 142 is situated below second pad 142. Fourth pad 156 is situated in the upper portion of lower layer 26 so that upper surface 164 of fourth pad 156 coincides with upper surface 30 of lower layer 26 in the area of fourth pad 156 and lower surface 162 of fourth pad 156 is positioned between lower surface 28 and upper surface 30 of lower layer 26.

Of course, any of pads 140, 142, 154 and 156 may be vertically positioned like pads 40 and 42 of insole 10, that is, essentially on the bottom of the insole. Likewise, pads 40 and 42 of insole 10 may be vertically positioned as are the pads of insole 100. At least one of pads 140, 142, 154 and 156, or a portion thereof, has a different rebound rate than at least one other of said pads or portion thereof, the other pad or portion being horizontally distinct from the one pad or portion thereof. Otherwise, each pad may have the same or different rebound rates, depending on the desired effect of insole 100. The elastomers and characteristics thereof used for pads 140, 142, 154 and 156 are described with respect to insole 10 above.

Any of pads 140, 142, 144 and 146 may be positioned like the other pads without departing from the spirit of the invention. Further, any pad may be positioned in upper layer 20 in a manner similar to the location of third pad 154 with respect to lower layer 26, that is, positioned below the upper surface and above the lower surface of a given layer or embedded so as to be completely encompassed by the given layer so that no portion of the pad extends vertically or horizontally outside the surfaces of that layer. In addition, any given pad or portion thereof may extend above the general level of the upper surface of the insole or below the general level of the lower surface of the insole.

The third embodiment of the present invention is shown in FIG. 9, the inventive concept being applied to sole 200 of a shoe instead of an insole. Sole 200 is part of a shoe 202, which also includes an insole 204 positioned above and resting on sole 200 and an upper 206 connected to sole 200 and generally positioned above sole 200, as is standard in the art.

Sole 200 has an upper surface 208 and a lower surface 210 and includes a first pad 212 positioned in an area analogous to metatarsus section 34 of insole 10 and a second pad 214 positioned rearwardly of first pad 212 in an area analogous to heel section 38 of insole 10. First pad 212 has an upper surface 216 and a lower surface 218 and second pad 214 has upper surface 220 and a lower surface 222. Upper surface 216 of first pad 212 coincides with upper surface 208 of sole 200 in the area of first pad 212. Upper surface 220 of second pad 214 coincides with upper surface 208 of sole 200 in the area of second pad 214. Lower surface 218 of first pad 212 and lower surface 222 of second pad 214 are situated between upper surface 208 and lower surface 210 of sole 200. First pad 212 and second pad 214 have different rebound rates as described in more detail with regard to insole 10. The elastomers and characteristics thereof used for pads 212 and 214 are described with respect to insole 10 above.

The fourth embodiment of the present invention is indicated generally at 300 in FIG. 10 and is similar to sole 200 except for the location of the pads. Sole 300 has an upper surface 308 and a lower surface 310 and includes a first pad 312 positioned in an area analogous to metatarsus section 34 of insole 10 and a second pad 314 positioned rearwardly of first pad 312 in an area analogous to heel section 38 of insole 10. First pad 312 has an upper surface 316 and a lower surface 318 and second pad 314 has upper surface 320 and a lower surface 322. First pad 312 and second pad 314 are embedded within sole 300. Thus, upper surface 316 and lower surface 318 of first pad 312 and upper surface 320 and lower surface 322 of second pad 314 are all positioned between upper surface 308 and lower surface 310 of sole 300. First pad 312 and second pad 314 have different rebound rates as described in more detail with regard to insole 10. The elastomers and characteristics thereof used for pads 312 and 314 are described with respect to insole 10 above.

Soles 200 and 300 illustrate only a pair of potential embodiments, just as insoles 10 and 100 illustrate only a pair of endless configurations. Any number of pads may be used with soles 200 and 300 without departing from the spirit of the invention. The pads of soles 200 and 300 may be positioned in any number of ways, as illustrated in regard to insoles 10 and 100 without departing from the spirit of the invention. No limitations are intended in regard to these matters. The primary key of the invention is that at least two pads have different rebound rates. Depending on the particular use, elastomers which vary the other characteristics may also be desireable. Thus, in addition to the various rebound rates, altering the hardness and/or the density will substantially improve the function of a given sole or insole.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the invention is an example and the invention is not limited to the exact details shown or described.

1. An insole comprising:
a first elastomeric pad and a second elastomeric pad horizontally distinct from one another, each pad having a rebound rate; and
the rebound rate of the first pad differing from the rebound rate of the second pad.

2. The insole of claim 1 wherein the first pad elastomer has a rebound rate ranging from 5.0% to 90.0%.

3. The insole of claim 2 wherein the second pad elastomer has a rebound rate ranging from 1.0% to 40.0%.

4. The insole of claim 3 wherein the first pad elastomer has a density ranging from 0.8 to 2.2.

5. The insole of claim 4 wherein the second pad elastomer has a density ranging from 0.7 to 2.0.

6. The insole of claim 5 wherein the first pad elastomer has a hardness with a durometer range of 30 to 70, Shore Hardness (00) scale.

7. The insole of claim 6 wherein the second pad elastomer has a hardness with a durometer range of 20 to 70, Shore Hardness (00) scale.

8. The insole of claim 5 wherein the first pad elastomer has a hardness with a durometer range of 35 to 70, Shore Hardness (00) scale.

9. The insole of claim 8 wherein the first pad elastomer has a hardness with a durometer range of 20 to 55, Shore Hardness (00) scale.

10. The insole of claim 1 wherein the insole has a forward and rearward orientation and the first pad is located forward of the second pad.

11. The insole of claim 10 wherein the first pad rebound rate is greater than the second pad rebound rate.

12. The insole of claim 11 wherein the insole has a forefoot section and a heel section and the first pad is located in the forefoot section and the second pad is located in the heel section.

13. The insole of claim 12 wherein the first pad has a density ranging from 0.8 to 2.2.

14. The insole of claim 13 wherein the second pad has a density ranging from 0.7 to 2.0.

15. The insole of claim 14 wherein first pad has a hardness with a durometer range of 30 to 70, Shore Hardness (00) scale.

16. The insole of claim 15 wherein the second pad has a hardness with a durometer range of 20 to 70, Shore Hardness (00) scale.

17. The insole of claim 14 wherein first pad has a hardness with a durometer range of 35 to 70, Shore Hardness (00) scale.

18. The insole of claim 17 wherein first pad has a hardness with a durometer range of 20 to 55, Shore Hardness (00) scale.

19. The insole of claim 1 wherein the insole has a lower surface and at least a portion of at least one of the pads forms part of the lower surface of the insole.

20. The insole of claim 1 wherein the insole has an upper surface and at least a portion of at least one of the pads forms part of the upper surface of the insole.

21. The insole of claim 1 wherein the insole has an upper surface and a lower surface and at least one of the pads extends from the upper surface of the insole to the lower surface of the insole.

22. The insole of claim 1 wherein the insole has an upper surface and a lower surface and at least one of the pads is disposed between the upper and lower surfaces of the insole.

23. The insole of claim 1 wherein the insole includes at least two layers and at least a portion of a pad is disposed between the upper and lower layers.

24. The insole of claim 1 wherein at least one of the first and second pads is a gel.

25. The insole of claim 1 wherein the first and second pads are carried by a carrier.

26. The insole of claim 25 wherein the carrier is at least partially formed of a material selected from a group consisting of a gel, a foam, an elastomer, leather and cloth.

27. A shoe sole comprising:
a first elastomeric pad and a second elastomeric pad horizontally distinct from one another, each pad having a rebound rate; and
the rebound rate of the first pad differing from the rebound rate of the second pad.

28. The sole of claim 27 wherein the first pad has a hardness with a durometer range of 30 to 70, Shore Hardness (00) scale.

29. The sole of claim 28 wherein the second pad has a hardness with a durometer range of 20 to 70, Shore Hardness (00) scale.

30. The sole of claim 29 wherein the first pad has a density ranging from 0.8 to 2.2.

31. The sole of claim 30 wherein the second pad has a density ranging from 0.7 to 2.0.

32. The sole of claim 31 wherein the first pad has a rebound rate ranging from 5.0% to 90.0%.

33. The sole of claim 32 wherein the second pad has a rebound rate ranging from 1.0% to 40.0%.
